SEQUENCE LISTING (26 sequences)

SEQ ID NO: 1 - DNA, 217 bp, mouse
ggacagcttctccagacctaggggtacaggtaaacactacaatattcaatttcacttttaaaacagtttgaaccaggcacagtggttcatgcttggaatcccaataagcaggaggatgaagcaggagaatcactgcaaactcaagaccagcctggtctacacataagttcacagagctacaaactagacatgtcattaagaaaaaaaaaaaaaaaaa

SEQ ID NO: 2 - DNA, 448 bp, mouse
ggacagcttctttagaatcccggcatttcctcctctacacctaatacgccacaggctgctaactttataagtggaacctactcaaaagcaacgtttctcaatccgtagatctagatccctttgggaggggtctaagagcatccgaaaacacagatattcatgttacattcataacagtagaaaaattacagttacaggtagtagcaaaataaattggggtcaccacaacatgaggaagagtattaaacagttgcagcattgagaaccactgctctaaaacaaatgtctctcaaattatcagaaagttgtgtgtttgtcctcggaaggatgggctgtggctcttccaatcttgcaaagtttgttcatggctgaccatgaaccaaaatgcctaaagcaattattatgacaactaccaactttttaaaaatttcaaaaaaaaaaaaaaaaa

SEQ ID NO: 3 - DNA, 349 bp, mouse
ggcccgcgtcgactagtactttttttttttttttttctgactttattgagaaataaagtcagaaaaaaaaaaaaccctgtaagacgtttgagggatctttaggagaaatacaaaggtgagaataggtgtcagataacgtggtatggttacttttcgtagacgaggtaaaagtattgggtttatatttccctaccatatgcatgcaaaagtttaagttgacaagtcataatgtctgcagcttattttaaattgaaactgatcagtgactgttgaattgtgatcaccccttaaacttgtctgtgcctatgtttaaatgtgataaaaattgaggcaaaaaaaaaaaaaaaaa

SEQ ID NO: 4 - DNA, 298 bp, mouse
ctagtactttttttttttttttttgcaagtgtagaaatttatttgaaccaacattttcactacaatgaaaatgacttcagcattgatccaagctcccacagcggagggtcaggaagtattgcactttgcttttttttttttttttttttttttggactccatacacggcaaaaggtttcaaaaaacattttgcttactttgttttacacactctcccaactcatgcacacactcattccaaaccaatgtttggttatccccaaaatagggaagaactccaaaaaaaaaaaaaaaaaaa

SEQ ID NO: 5 - DNA, 399 bp, mouse
ttttttttttttgcagagcttaaaatgtattgacattgtgaagggacaacttggagacaggccaagcctgttgagatgacgcacggttaagacatgctgattctcaggatggcgtcagaagggttaaccaacatggctggaacttggcattgtgcattttcattagcaggggatcactactctaaagtttctcctagagtatcgtaatggctgcctgcagaacaactacagacctaatttagatgttaaagttgatcagaattatcagcaacctggtttgcatttggtcactgctgcggggcgaggaggtaagttttcttaatggattccttgtttatatgtttagccaccaactgactccagagaataatgaaaaataagttaggaaaaaaaaaaaaa

SEQ ID NO: 6 - DNA, 388 bp, mouse
ttttttttttttgcggggagatggtgcctttttattaaggttgacatcaagcaaagcactaagaaaggatacacagaggagggaaggggcacacacagtcaccacttcggaaggtaggagcacatcagttaaggacactcaggacagccacatgctccgtgccctgggttgaggagtggggcataaaagggaaaagagcagaaagcgccatgatagcctggggcttgcagacgggtctgaagccccctgaacctaatactagagccacaagccctgcccctgagggctcacacactactacacaagtggacacacataacacaagacattatggaggcaacaccagaaagcagagggtaccgacatattgacaggaaaaaaaaaaaaa

SEQ ID NO: 7 - DNA, 399 bp, mouse
tttttttttttttgcagagcttaaaatgtattgacattgtgaagggacaacttggagacaggccaagcctgttgagatgacgcacggttaagacatgctgattctcaggatggcgtcagaagggttaaccaacatggctggaacttggcattgtgcattttcattagcaggggatcactactctaaagtttctcctagagtatcgtaatggctgcctgcagaacaactacagacctaatttagatgttaaagttgatcagaattatcagcaacctggtttgcatttggtcactgctgcggggcgaggaggtaagttttcttaatggattccttgtttatatgtttagccaccaactgactccagagaataatgaaaaataagttaggaaaaaaaaaaaa

SEQ ID NO: 8 - DNA, 356 bp, mouse
gattggacagcttcacaatggtaaataggagtatcttaggccacactgaggcttctgacaggcagtgaataattcaagaccatggttcttaggtgaagttaaattttacatgactcacatgtgatggaacaaaaaaaaatctcatatcaaccacagtgttcttcaattcagcccttttataaattaatttctcaacttgtttgctgtatttatgaaatgaacctaagctacccttcttatgatgagactgtatcaaataatcaccatgaaaattacagcatactttgatatataagacaatcatttgtacatgacgtatttaactaaaaaaaaaaaaaaaaaaaaaaaaaaaaaaa

SEQ ID NO: 9 - DNA, 498 bp, mouse
  misc_feature (496)..(496): base 496 is unidentified
ggacagcttcttcatcttctgataatttcccttttccaactagttgacttcctaactgtagaacattaccaacaagtccttgatgagatgtcagatccatcatgaatccctcatgagcatgaaactgttctttcacttcttcaacatcatttgaaatctctccttgtgctcgcaatgtatcctcggcagaaagaagccatgaaagtacttcttctaaagcagtttggtaactatccagatttacttccgtctccatcaatgaactgtcaagtgacttgtctctgggagcttccaaatgctgtgaaggataggggctctgtgtggaatcagaggtggcaacataagcagcctgtgtggaggcataactcttgaatcgaggcttaggagatgaagaagtttgttcatagccctgtgctagactgactgtgatctgttgagagtaatgcatctggtgatgtaattgaaaatgttcttctctagttacttttgaagctgncc

SEQ ID NO: 10 - DNA, 421 bp, mouse
ggacagcttcagaaagtcattgtgttctgggttctccacctctacaacaccccatgggtagagacggcctctaaccttcttgcctttggcttcaatcaactggttggagccaaccacagagaatgggatactggccttgaggagtctagtctgctccttaaagtcttcatcttcatctgactctgcatcaggtaagtgatagattttaatgctatgctcttcatagaaattcactattaagagtatacatgttgactgattcttagaaataaattctaacattagtggttcaggaggaataaaatatcagagttttccttacaaaatttaaagtgtaacaatgaaatataaatgttaaaagtacttcataccaaaggagttggggggttgagggcagaaaaagaaggaaaaaaaaaaaaaa

SEQ ID NO: 11 - DNA, 299 bp, mouse
tttttttttttttttttatcatagaacctttattatttttcctctaaattcttagaaacgtacatgacgcccgtgttctagctgtgattccatttgatagacatttaaaacacatcttcatgtataaatattttacatttggtccatagaacagataattttactaaataatactgtaatttttttaaaacaggctctgtatatagtctgaataggcatatattacatatacttttatatagagataaatttacttggtgttctgagaataaatccttattgcaaaaaaaaaaaaaaaa

SEQ ID NO: 12 - DNA, 20 bp, mouse
taacaacaacagcccatcca

SEQ ID NO: 13 - DNA, 20 bp, mouse
ggagtaaccctgctccatga

SEQ ID NO: 14 - DNA, 24 bp, mouse
ggaggggtctaagagcatccgaaa

SEQ ID NO: 15 - DNA, 23 bp, mouse
aagattggaagagccacagccca

SEQ ID NO: 16 - DNA, 20 bp, mouse
gagtttgtgctgtcagtggc

SEQ ID NO: 17 - DNA, 20 bp, mouse
ctcccctggctgctatactg

SEQ ID NO: 18 - DNA, 20 bp, mouse
tttggtcttccccttttcct

SEQ ID NO: 19 - DNA, 20 bp, mouse
aagactccactcaaccccct

SEQ ID NO: 20 - DNA, 20 bp, mouse
aagacgtttgagggatcttt

SEQ ID NO: 21 - DNA, 20 bp, mouse
ttaaggggtgatcacaattc

SEQ ID NO: 22 - DNA, 20 bp, mouse
tgagatgacgcacggttaag

SEQ ID NO: 23 - DNA, 20 bp, mouse
gaaaacttacctcctcgccc

SEQ ID NO: 24 - DNA, 24 bp, mouse
gaacatcatccctgcctctactgg

SEQ ID NO: 25 - DNA, 24 bp, mouse
aatgccagccccagcgtcaaaggt

SEQ ID NO: 26 - protein, 72 aa, mouse MC1-peptide
VAEGSRPTCTFVVINLKVKLVTQVLCLPEHKSDWYAPPHLLLIVAFELGAQDVCLNVSSCVLCTMLFFFFFF

(19) United States
(12) Patent Application Publication
(10) Pub. No.: US 2005/0267050 A1
(43) Pub. Date: Dec. 1, 2005

(54) Compositions and methods for treating mdma-induced toxicity

(75) Inventors: Rabi Simantov, Rehovot (IL); Weiping Peng, Lincoln, NE (US)

Correspondence Address: WINSTON & STRAWN LLP, 1700 K STREET, N.W., WASHINGTON, DC, 20006, US

(73) Assignee: YEDA RESEARCH AND DEVELOPMENT CO. LTD., Rehovot (IL)

(21) Appl. No.: 10/508,424

(22) PCT Filed: Mar. 13, 2003
(86) PCT No.: PCT/IL03/00214

§ 371 Date: May 9, 2005

Related U.S. Application Data
(60) Provisional application No. 60/364,603, filed on Mar. 18, 2002.

(51) Int. Cl. A61K 31/704

(52) U.S. Cl. 514/26

(57) ABSTRACT

This invention relates to methods and compositions for treating or preventing the symptoms associated with toxicity induced by ingestion of MDMA. In one aspect, the present invention is related to methods for treating or preventing the symptoms associated with MDMA, or MDMA-related psychoactive drugs by administering to a subject in need thereof a composition that includes a therapeutically effective amount of an agent which inhibits γ-amino butyric acid (GABA) transporter activity or reduces GABA transporter level in the brain.

### CROSS-REFERENCE TO RELATED APPLICATIONS

This application is a continuation of International application PCT/IL03/00214 filed Mar. 13, 2003, which claims the benefit of provisional application 60/364,603 filed Mar. 18, 2002, the entire content of each of which is expressly incorporated herein by reference thereto.

### FIELD OF THE INVENTION

This invention relates to methods and compositions for treating or preventing the symptoms associated with toxicity induced by ingestion of methylenedioxymethamphetamine (hereinafter “MDMA”).

### BACKGROUND OF THE INVENTION

3,4-Methylenedioxymethamphetamine (hereinafter “MDMA” or “Ecstasy”) is a widely abused recreational drug. It is known to cause psychostimulation and can induce hallucination and long-term neuropsychiatric behaviors, including depression and psychosis (for review, see Green et al., 1995). Initially it was found that MDMA is neurotoxic, primarily to serotonergic neurons, and induces degeneration of neuronal fibers in rodents and primates. Physiologically, MDMA induces severe hyperthermia, occasionally resulting in acute toxicity and death (Hegadoren et al., 1999). Long-term memory deficits were observed in rats exposed to the drug during brain development. In humans, MDMA was reported to impair verbal, visual and recall memories (Zakzanis and Young, 2001). The drug also induces programmed cell death in cultured human JAR cells (Simantov and Tauber, 1997) and rat neocortical neurons.

A characteristic feature of MDMA activity in vivo is the wide range of effects it induces which include psychostimulation, hallucination, hyperthermia, memory loss and long-lasting changes in behavior (Green et al., 1995 and Hegadoren et al., 1999). Evidence indicates that MDMA binds to serotonin transporters (SERT), blocks serotonin uptake and enhances serotonin exchange and release. MDMA also modulates the activity of dopaminergic (Shankaran and Gudelsky, 1998), y-amino butyric acid (GABA) (Yamamoto et al., 1995) and glutamate (FINNEGAN and Taraska, 1996) neurons. It was also documented that MDMA induces changes in expression of genes such as C-FOS and BCL-X (Stephenson et al., 1999), but a causal association between these or other genes and a certain behavior induced by the drug has not been disclosed.

There exists a long-felt need for an effective means of treating and preventing the symptoms associated with toxicity of MDMA or MDMA-related drugs. None of the background art discloses the use of inhibitors of GABA reuptake for treating the symptoms associated with ingestion of MDMA.

### SUMMARY OF THE INVENTION

The present invention provides compositions and methods for treating or preventing the symptoms associated with the ingestion of methylenedioxymethamphetamine (MDMA) or MDMA-related psychoactive drugs. In one aspect, the present invention is related to methods for treating or preventing the symptoms associated with MDMA, or MDMA-related psychoactive drugs comprising administering to a subject in need thereof a composition comprising a therapeutically effective amount of an agent which inhibits γ-amino butyric acid (GABA) transporter activity or reduces GABA transporter levels in the brain.

The present invention is based in part on the unexpected discovery that administration of MDMA up-regulates the expression of various GABA transporter genes, specifically GABA transporter 1 (GAT1) and GABA transporter 4 (GAT4) within the brain. Accordingly, inhibiting the activity or reducing the level of GABA transporter in the brain is advantageous in treating subjects suffering from symptoms associated with MDMA or MDMA-related drugs.

In one embodiment, the present invention relates to a pharmaceutical composition comprising an effective amount of a GABA transporter inhibitor for treating and preventing the symptoms associated with the ingestion of MDMA.

In another embodiment, the present invention is related to a method for treating or preventing the symptoms associated with the ingestion of MDMA comprises administering to a subject in need thereof a composition comprising a therapeutically effective amount of a GABA transporter inhibitor, capable of reducing the activity of GABA transporter in the brain. In certain preferred embodiments, a GABA transporter inhibitor is selected from Tiagabine, nipecotic acid derivatives such as for example NO-711, and Vigabatrin.

In another embodiment, the method of the present invention comprises administering to a subject in need thereof a therapeutically effective amount of a nucleic acid molecule capable of reducing the expression of GABA transporter in the brain, in sufficient amount and duration to inhibit symptoms associated with MDMA in said subject. In a preferred embodiment, antisense RNA, or dsRNA are used for reducing the expression of GABA transporters.

In another embodiment the present invention provides oligonucleotide sequences capable of binding the GAT1 or GAT4 gene sequences and inhibiting its expression. These oligonucleotide sequences are capable of specifically inhibiting the expression of GAT1 or GAT4.

Preferred molecules according to the present invention are oligonucleotide antisense molecules derived from or complementary to the GAT1 or GAT4 mRNA. Preferred oligonucleotide sequences have a length of about 5 to about 40 nucleotides, more preferred molecules are 10-30 nucleotides long, and most preferred ones are 15-25 long. Specifically preferred molecules are DNA sequences, other molecules which are part of the present invention are for example RNA and PNA (peptide nucleic acid) molecules.

Other preferred molecules according to the present invention are short duplex RNAs corresponding to the sequence of GAT1 or GAT4 which inhibit GAT1 or GAT4 expression via RNA interference mechanism (RNAi). In one embodiment, the short duplex RNAs are synthetic molecules. In another embodiment, an expression vector comprising short duplex RNAs is used.

Most preferably antisense DNA or dsRNA sequences are derived from the human or mouse GAT1 or GAT4 mRNA sequences. The sequence of GAT1 is disclosed in Science, 249, 1303-1306, 1990, and in FEBS Lett. 269, 181-184, 1990. The sequence of GAT4 is disclosed in J. Biol. Chem. 268, 2106-2112. U.S. Pat. No. 6,225,115 discloses the cDNA sequence of GAT-1 which may be used in the present invention for preparing antisense DNA or dsRNA sequences suitable for inhibiting GAT-1 expression.

In yet another embodiment, the method of the present invention comprises administering to a subject in need thereof a composition comprising a therapeutically effective amount of an anti-GABA transporter antibodies, said antibodies being capable of reducing the activity of GABA transporter in the brain, in sufficient amount and duration to inhibit symptoms associated with MDMA in said subject. In a preferred embodiment, anti-GAT1 or anti-GAT4 monoclonal antibodies are used in order to reduce the activity of GAT1 or GAT4 in the brain.

MDMA-related psychoactive drugs, the effects of which are treated by the method of the present invention, include for example methamphetamine, 3,4-methylenedioxyamphetamine (MDA) and similar psychoactive abused drugs such as cocaine.

The present invention is also related to a method of attenuating the hyperthermic effects of MDMA or MDMA-related drugs comprising administering to a subject in need thereof a therapeutic composition comprising a therapeutically effective amount of an agent which inhibits GABA reuptake, thereby attenuating the hyperthermic effects associated with MDMA or MDMA-related drugs and similar psychostimulants.

The pharmaceutical compositions of the present invention may be administered in a number of ways depending upon whether local or systemic treatment is desired and upon the area to be treated. Administration may be topical, oral or parenteral. Parenteral administration includes intravenous drip, subcutaneous, intraperitoneal or intramuscular injection, pulmonary administration, e.g., by inhalation or insufflation, or intracranial, e.g., intrathecal or intraventricular, administration. Oligonucleotides with at least one 2′-O-methoxyethyl modification are believed to be particularly useful for oral administration.

These and further embodiments will be apparent from the detailed description and examples that follow.

### BRIEF DESCRIPTION OF THE DRAWINGS

FIG. 1. Differential display (DD) PCR analysis of mRNA from the frontal cortex (FC) or midbrain (MB) of control (saline) or MDMA treated mice. A—Complete DNA sequencing acrylamide gel of a representative control (−) and MDMA-treated (+) samples. B and C depict the DD-PCR expression of six cDNAs differentially expressed upon MDMA treatment.

FIG. 2. RT-PCR analysis of mGAT1, mGAT2 and mGAT4 (A), and three unknown differentially expressed cDNAs isolated from MDMA-treated mice (B).

FIG. 3. Time course of mGAT1, mGAT2 and mGAT4 expression in mice treated with MDMA for 2 hours-7 days, using semi-quantitative RT-PCR, and GAPDH as a standard.

FIG. 4. Real-time PCR analysis of mGAT1 and mGAT4, and GAPDH as a control. A—Polymerization curves of GAPDH in RNA samples from saline (−) or MDMA (+) treated midbrain. B—Polymerization of 4 concentrations of a standard DNA. C—Polymerization curves of mGAT1 in RNA samples from saline (−) or MDMA (+) treated midbrain. D—Melting curves of mGAT1 DNA products shown in FIG. 4C. E—Polymerization curves of mGAT4 in RNA samples from saline (−) or MDMA (+) treated midbrain. F—Melting curves of mGAT4 DNA products shown in FIG. 4E.

FIG. 5. Western immunoblotting of mGAT1 in wild type (+/+) and serotonin transporter knockout (−/−) mice.

FIG. 6. Effect of administration of Tiagabine, NO-711, Vigabatrin, guvacine and valproate on the survival of mice treated with a toxic dose of MDMA.

### DETAILED DESCRIPTION OF THE INVENTION

The term “inhibit GABA reuptake” as used herein includes the uptake of GABA by the GABA nerve terminal, glial cells and any GABA cell surface transporter.

The term “MDMA-related drugs” as used herein includes any pharmacologically and structurally MDMA-related compounds.

The present invention teaches a novel approach to restrain damaging effects of MDMA or MDMA-related drugs, including acute toxicity and hyperthermia by reducing the activity of specific GABA transporters which are up-regulated in response to MDMA or pharmacologically and structurally related compounds.

The present invention is related to methods for treating or preventing the symptoms associated with MDMA or MDMA-related drugs comprising administering to a subject in need thereof a composition comprising a therapeutically effective amount of an agent which inhibits GABA reuptake. The inhibition of GABA reuptake is obtained preferably by inhibiting GABA transporter activity using specific inhibitors or blockers.

The present invention is based on part on the unexpected discovery that administration of MDMA up-regulates the expression of various GABA transporter genes, specifically GABA transporter 1 (GAT1) and GABA transporter 4 (GAT4) within the brain. Using the differential display polymerase chain reaction (DD-PCR) method, the inventors of the present invention unexpectedly found an induction of GAT1 and GAT4 mRNA and protein levels following MDMA exposure.

Specifically, changes in gene expression were found in the brain of mice treated with MDMA. Frontal cortex and midbrain mRNA, analyzed using the differential display polymerase chain reaction (DD-PCR) method, showed an altered expression of several cDNAs, 11 of which were isolated, cloned and sequenced. The sequence of one MDMA-induced mRNA corresponds (99.3%) to the mouse γ-amino butyric acid (GABA) transporter 1 (mGAT1). Semi-quantitative PCR analysis with primers selective to the reported mGAT1 sequence confirmed that MDMA treatment increased mGAT1 expression. Time-course study of the expression of the GABA transporter subtypes showed that MDMA induced a differential temporal activation of subtypes mGAT1 and mGAT4, but had no effect on mGAT2. Quantitative Real-time PCR (QRT-PCR) further proved the increased expression of mGAT1 and mGAT4 upon MDMA treatment. Western immunoblotting with anti-GAT-1 antibodies showed that MDMA also increased mGAT1 protein levels, suggesting that neurotransmission of GABA was altered.

In a preferred embodiment, specific GABA transporter inhibitors are used to reduce the transporter activity. The GABA uptake system has traditionally been classified as either neuronal or glial GABA uptake carriers, on the basis of pharmacological selectivity for specific GABA uptake inhibitors.

Four subtypes of the rat and mouse GABA uptake carrier, whose pharmacology cannot be totally explained by the traditional neuronal and glial GABA uptake carriers were cloned, of which GAT1 and GAT4 are relevant to the present invention. Gaustella et al., (1990, Science, 249, 1303-1306) and Nelson et al. (1990, FEBS Lett. 269, 181-184) reported the cloning of GAT1, which appears to be a neuronal GABA uptake carrier due to its high sensitivity to nipecotic acid. GAT4 (Liu et al., 1993, J. Biol. Chem. 268, 2106-2112; also termed GAT-B by Clark et al., (1992, Neuron 9, 337-348) is highly enriched in the brain stem, but not present in the cerebellum or cerebral cortex.

A well-known and potent inhibitor of GABA uptake from the synaptic cleft into presynaptic nerve terminals and glial cells is, for example, 3-piperidinecarboxylic acid (nipecotic acid). However, being a relatively polar compound and therefore unable to cross the blood-brain barrier, 3-piperidinecarboxylic acid itself has found no practical utility as a drug.

Examples of GABA uptake inhibitors which can be used in the present invention are disclosed for example in U.S. Pat. No. 4,383,999 and U.S. Pat. No. 4,514,414 and in EP 236342 as well as in EP 231996 in which some derivatives of N-(4,4-disubstituted-3-butenyl)azaheterocyclic carboxylic acids are claimed as inhibitors of GABA uptake. In EP 342635 and EP 374801, N-substituted azaheterocyclic carboxylic acids in which an oxime ether group and vinyl ether group forms part of the N-substituent respectively are claimed as inhibitors of GABA uptake. Further, in WO 91/07389 and WO 92/20658, N-substituted azacyclic carboxylic acids are claimed as inhibitors of GABA uptake. EP 221572 claims that 1-aryloxyalkylpyridine-3-carboxylic acids are inhibitors of GABA uptake.

Other GAT1 inhibitors which can be used in the present invention are N-(4,4-diphenyl-3-buten-1-yl)nipecotic acid (designated SK&F 89976A), N-(4,4-diphenyl-3-buten-1-yl)guvacine (designated SK&F 100330A), N-(4,4-diphenyl-3-buten-1-yl)-homo-.beta.-proline (designated SK&F 100561) and N-(4-phenyl-4-(2-thienyl)-3-buten-1-yl)nipecotic acid (designated SK&F 100604J) which are orally active inhibitors of GABA uptake.

In another embodiment, specific nucleic acid molecules are used to reduce the expression level of GABA transporters, preferably GAT1 or GAT4, most preferably the human GAT1 or GAT4. Most preferable antisense DNA or dsRNA sequences derived from the human or mouse GAT1 or GAT4 mRNA sequence are used to reduce the expression level of GABA transporters. The sequence of GAT1 is disclosed in Science, 249, 1303-1306, 1990, and in FEBS Lett. 269, 181-184, 1990. The sequence of GAT4 is disclosed in J. Biol. Chem. 268, 2106-2112. U.S. Pat. No. 6,225,115 discloses the mRNA sequence of GAT-1 which may be used in the present invention for preparing antisense DNA or dsRNA sequences suitable for inhibiting GAT-1 expression.

Thus, in one preferred embodiment, this invention provides a pharmaceutical composition comprising an effective amount of specific antisense molecules effective to reduce the expression of GAT1 or GAT4 transporters by binding specifically with mRNA encoding GAT1 or GAT4 transporters in the cell so as to prevent its translation. The pharmaceutical composition further comprising a pharmaceutically acceptable hydrophobic carrier capable of passing through a cell membrane.

The antisense molecules used in the present invention are preferably synthetic antisense oligonucleotides which are designed to bind to mRNA encoding specific GABA transporters and are useful as drugs to inhibit expression of GABA transporter genes in patients. In a preferred embodiment, the synthetic antisense oligonucleotides designed to recognize and selectively bind to mRNA, are constructed to be complementary to portions of the nucleotide sequences of GAT1 or GAT4 mRNA.

Use of antisense oligonucleotides is well known in the art. For example U.S. Pat. No. 6,228,642 describes methodology and terminology related to antisense technology. This patent is incorporated here in its entirety by reference. The following text includes non-limitative examples for such methods and terminology.

In accordance with this invention, persons of ordinary skill in the art will understand that messenger RNA includes not only the information to encode a protein using the three letter genetic code, but also associated ribonucleotides which form a region known to such persons as the 5′-untranslated region, the 3′-untranslated region, the 5′ cap region and intron/exon junction ribonucleotides. Thus, oligonucleotides may be formulated in accordance with this invention which are targeted wholly or in part to these associated ribonucleotides as well as to the informational ribonucleotides. The oligonucleotide may therefore be specifically hybridizable with a transcription initiation site region, a translation initiation codon region, a 5′ cap region, an intron/exon junction, coding sequences, a translation termination codon region or sequences in the 5′- or 3′-untranslated region. Since, as is known in the art, the translation initiation codon is typically 5′-AUG (in transcribed mRNA molecules; 5′-ATG in the corresponding DNA molecule), the translation initiation codon is also referred to as the “AUG codon,” the “start codon” or the “AUG start codon.” A minority of genes have a translation initiation codon having the RNA sequence 5′-GUG, 5′-UUG or 5′-CUG, and 5′-AUA, 5′-ACG and 5′-CUG have been shown to function in vivo. Thus, the terms “translation initiation codon” and “start codon” can encompass many codon sequences, even though the initiator amino acid in each instance is typically methionine (in eukaryotes) or formylmethionine (prokaryotes). It is also known in the art that eukaryotic and prokaryotic genes may have two or more alternative start codons, any one of which may be preferentially utilized for translation initiation in a particular cell type or tissue, or under a particular set of conditions. In the context of the invention, “start codon” and “translation initiation codon” refer to the codon or codons that are used in vivo to initiate translation of an mRNA molecule transcribed from a gene encoding GAT1 or GAT4, regardless of the sequence(s) of such codons.

It is also known in the art that a translation termination codon (or “stop codon”) of a gene may have one of three sequences, i.e., 5′-UAA, 5′-UAG and 5′-UGA (the corresponding DNA sequences are 5′-TAA, 5′-TAG and 5′-TGA, respectively). The terms “start codon region,” “AUG region” and “translation initiation codon region” refer to a portion of such an mRNA or gene that encompasses from about 25 to about 50 contiguous nucleotides in either direction (i.e., 5′ or 3′) from a translation initiation codon. This region is a preferred target region. Similarly, the terms “stop codon region” and “translation termination codon region” refer to a portion of such an mRNA or gene that encompasses from about 25 to about 50 contiguous nucleotides in either direction (i.e., 5′ or 3′) from a translation termination codon. This region is a preferred target region. The open reading frame (ORF) or “coding region,” which is known in the art to refer to the region between the translation initiation codon and the translation termination codon, is also a region which may be targeted effectively. Other preferred target regions include the 5′ untranslated region (5′UTR), known in the art to refer to the portion of an mRNA in the 5′ direction from the translation initiation codon, and thus including nucleotides between the 5′ cap site and the translation initiation codon of an mRNA or corresponding nucleotides on the gene and the 3′ untranslated region (3′UTR), known in the art to refer to the portion of an mRNA in the 3′ direction from the translation termination codon, and thus including nucleotides between the translation termination codon and 3′ end of an mRNA or corresponding nucleotides on the gene. The 5′ cap of an mRNA comprises an N7-methylated guanosine residue joined to the 5′-most residue of the mRNA via a 5′—5′ triphosphate linkage. The 5′ cap region of an mRNA is considered to include the 5′ cap structure itself as well as the first 50 nucleotides adjacent to the cap. The 5′ cap region may also be a preferred target region.

Although some eukaryotic mRNA transcripts are directly translated, many contain one or more regions, known as “introns,” which are excised from a pre-mRNA transcript to yield one or more mature mRNAs. The remaining (and therefore translated) regions are known as “exons” and are spliced together to form a continuous mRNA sequence. mRNA splice sites, i.e., exon-exon or intron-exon junctions, may also be preferred target regions, and are particularly useful in situations where aberrant splicing is implicated in disease, or where an overproduction of a particular mRNA splice product is implicated in disease. Aberrant fusion junctions due to rearrangements or deletions are also preferred targets. Targeting particular exons in alternatively spliced mRNAs may also be preferred. It has also been found that introns can also be effective, and therefore preferred, target regions for antisense compounds targeted, for example, to DNA or pre-mRNA.

Once the target site or sites have been identified, oligonucleotides are chosen which are sufficiently complementary to the target, i.e., hybridize sufficiently well and with sufficient specificity, to give the desired modulation. “Hybridization”, in the context of this invention, means hydrogen bonding, also known as Watson-Crick base pairing, between complementary bases, usually on opposite nucleic acid strands or two regions of a nucleic acid strand. Guanine and cytosine are examples of complementary bases which are known to form three hydrogen bonds between them. Adenine and thymine are examples of complementary bases which form two hydrogen bonds between them. “Specifically hybridizable” and “complementary” are terms which are used to indicate a sufficient degree of complementarity such that stable and specific binding occurs between the DNA or RNA target and the oligonucleotide.

It is understood that an oligonucleotide need not be 100% complementary to its target nucleic acid sequence to be specifically hybridizable. An oligonucleotide is specifically hybridizable when binding of the oligonucleotide to the target interferes with the normal function of the target molecule to cause a loss of utility, and there is a sufficient degree of complementarity to avoid non-specific binding of the oligonucleotide to non-target sequences under conditions in which specific binding is desired, i.e., under physiological conditions in the case of in vivo assays or therapeutic treatment or, in the case of in vitro assays, under conditions in which the assays are conducted.

Hybridization of antisense oligonucleotides with mRNA interferes with one or more of the normal functions of mRNA. The functions of mRNA to be interfered with include all vital functions such as, for example, translocation of the RNA to the site of protein translation, translation of protein from the RNA, splicing of the RNA to yield one or more mRNA species, and catalytic activity which may be engaged in by the RNA. Binding of specific protein(s) to the RNA may also be interfered with by antisense oligonucleotide hybridization to the RNA.

In the context of this invention, the term “oligonucleotide” refers to an oligomer or polymer of ribonucleic acid or deoxyribonucleic acid. This term includes oligonucleotides composed of naturally-occurring nucleobases, sugars and covalent intersugar (backbone) linkages as well as oligonucleotides having non-naturally-occurring portions which function similarly. Such modified or substituted oligonucleotides are often preferred over native forms because of desirable properties such as, for example, enhanced cellular uptake, enhanced binding to target and increased stability in the presence of nucleases.

The antisense compounds in accordance with this invention preferably comprise from about 5 to about 50 nucleobases. Particularly preferred are antisense oligonucleotides comprising from about 8 to about 30 nucleobases (i.e. from about 8 to about 30 linked nucleosides). As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn the respective ends of this linear polymeric structure can be further joined to form a circular structure, however, open linear structures are generally preferred. Within the oligonucleotide structure, the phosphate groups are commonly referred to as forming the internucleoside backbone of the oligonucleotide. The normal linkage or backbone of RNA and DNA is a 3′ to 5′ phosphodiester linkage.

Specific examples of preferred antisense compounds useful in this invention include oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides.

Preferred modified oligonucleotide backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included. The preparation of the above phosphorus-containing linkages is disclosed for example in U.S. Pat. No. 5,625,050.

Preferred modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. The preparation of the above oligonucleosides is disclosed for example in U.S. Pat. No. 5,677,439.

In other preferred oligonucleotide mimetics, both the sugar and the internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative The preparation of the above oligonucleosides mimetics is disclosed for example in U.S. Pat. No. 5,719,262. Further teaching of PNA compounds can be found in Nielsen et al. (Science, 1991, 254, 1497-1500).

Most preferred embodiments of the invention are oligonucleotides with phosphorothioate backbones and oligonucleosides with heteroatom backbones, and in particular —CH₂—NH—O—CH₂—, —CH₂—N(CH₃)—O—CH₂— [known as a methylene (methylimino) or MMI backbone], —CH₂O—N(CH₃)CH₂—, —CH₂—N(CH₃)—N(CH₃)—CH₂— and —O—N(CH₃)—CH₂—CH₂— [wherein the native phosphodiester backbone is represented as —O—P—O—CH₂—] of the above referenced U.S. Pat. No. 5,489,677, and the amide backbones of the above referenced U.S. Pat. No. 5,602,240. Also preferred are oligonucleotides having morpholino backbone structures of the above-referenced U.S. Pat. No. 5,034,506.

Modified oligonucleotides may also contain one or more substituted sugar moieties. Preferred oligonucleotides comprise one of the following at the 2′ position: OH; F; O—, S—, or N-alkyl, O-alkyl-O-alkyl, O—, S—, or N-alkenyl, or O—, S— or N-alkynyl, wherein the alkyl, alkenyl and alkynyl may be substituted or unsubstituted C1 to C10 alkyl or C2 to C10 alkenyl and alkynyl. Particularly preferred are O[(CH₂)ₙO]ₘCH₃, O(CH₂)ₙOCH₃, O(CH₂)₂ON(CH₃)₂, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. Other preferred oligonucleotides comprise one of the following at the 2′ position: C1 to C10 lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A preferred modification includes 2′-methoxyethoxy (2′-O—CH₂CH₂OCH₃, also known as 2′-O-(2-methoxyethyl) or 2′-MOE) (Martin et al., Helv. Chim. Acta 1995, 78, 486-504) i.e., an alkoxyalkoxy group.

Other preferred modifications include 2′-methoxy (2′-O-CH₃), 2′-aminopropoxy (2′-OCH₂CH₂CH₂NH₂) and 2′-fluoro (2′-F). Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar. Representative United States patent that teaches the preparation of such modified sugars structures is U.S. Pat. No. 5,700,920.

Oligonucleotides may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C or m5c), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further nucleobases include those disclosed in U.S. Pat. No.3,687,808, those disclosed in the Concise Encyclopedia Of Polymer Science And Engineering 1990, pages 858-859, Kroschwitz, J. I., ed. John Wiley & Sons, those disclosed by Englisch et al. (Angewandte Chemie, International Edition 1991, 30, 613-722), and those disclosed by Sanghvi, Y. S., Crooke, S. T. and Lebleu, B., eds., Antisense Research and Applications 1993, CRC Press, Boca Raton, pages 289-302. Certain of these nucleobases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-Methylcytosine substitutions have been shown to increase nucleic acid duplex stability by 0.6-1.2.degree. C. (Sanghvi, Y. S., Crooke, S. T. and Lebleu, B., eds., Antisense Research and Applications 1993, CRC Press, Boca Raton, pages 276-278) and are presently preferred base substitutions, even more particularly when combined with 2′-O-methoxyethyl sugar modifications. Representative United States patent that teaches the preparation of certain of the above noted modified nucleobases as well as other modified nucleobases is U.S. Pat. No. 5,681,941.

Another modification of the oligonucleotides of the invention involves chemically linking to the oligonucleotide one or more moieties or conjugates which enhance the activity, cellular distribution or cellular uptake of the oligonucleotide. Such moieties include but are not limited to lipid moieties such as a cholesterol moiety (Letsinger et al., Proc. Natl. Acad. Sci. USA 1989, 86, 6553-6556), cholic acid (Manoharan et al., Bioorg. Med. Chem. Lett. 1994, 4, 1053-1059), a thioether, e.g., hexyl-S-tritylthiol (Manoharan et al., Ann. N.Y. Acad. Sci. 1992, 660, 306-309; Manoharan et al., Bioorg. Med. Chem. Let. 1993, 3, 2765-2770), a thiocholesterol (Oberhauser et al., Nucl. Acids Res. 1992, 20, 533-538), an aliphatic chain, e.g., dodecandiol or undecyl residues (Saison-Behmoaras et al., EMBO J. 1991, 10, 1111-1118; Kabanov et al., FEBS Lett. 1990, 259, 327-330; Svinarchuk et al., Biochimie 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett. 1995, 36, 3651-3654; Shea et al., Nucl. Acids Res. 1990, 18, 3777-3783), a polyamine or a polyethylene glycol chain (Manoharan et al., Nucleosides & Nucleotides 1995, 14, 969-973), or adamantane acetic acid (Manoharan et al., Tetrahedron Lett. 1995, 36, 3651-3654), a palmityl moiety (Mishra et al., Biochim. Biophys. Acta 1995, 1264, 229-237), or an octadecylamine or hexylamino-carbonyl-oxycholesterol moiety (Crooke et al., J. Pharmacol. Exp. Ther. 1996, 277, 923-937).

The present invention also includes oligonucleotides which are chimeric oligonucleotides. “Chimeric” oligonucleotides or “chimeras,” in the context of this invention, are oligonucleotides which contain two or more chemically distinct regions, each made up of at least one nucleotide. These oligonucleotides typically contain at least one region wherein the oligonucleotide is modified so as to confer upon the oligonucleotide increased resistance to nuclease degradation, increased cellular uptake, and/or increased binding affinity for the target nucleic acid. An additional region of the oligonucleotide may serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of antisense inhibition of gene expression. Cleavage of the RNA target can be routinely detected by gel electrophoresis and, if necessary, associated nucleic acid hybridization techniques known in the art. This RNAse H-mediated cleavage of the RNA target is distinct from the use of ribozymes to cleave nucleic acids. Ribozymes are not comprehended by the present invention.

Examples of chimeric oligonucleotides include but are not limited to “gapmers,” in which three distinct regions are present, normally with a central region flanked by two regions which are chemically equivalent to each other but distinct from the gap. A preferred example of a gapmer is an oligonucleotide in which a central portion (the “gap”) of the oligonucleotide serves as a substrate for RNase H and is preferably composed of 2′-deoxynucleotides, while the flanking portions (the 5′ and 3′ “wings”) are modified to have greater affinity for the target RNA molecule but are unable to support nuclease activity (e.g., fluoro- or 2′-O-methoxyethyl-substituted). Chimeric oligonucleotides are not limited to those with modifications on the sugar, but may also include oligonucleosides or oligonucleotides with modified backbones, e.g., with regions of phosphorothioate (P.dbd.S) and phosphodiester (P.dbd.O) backbone linkages or with regions of MMI and P.dbd.S backbone linkages. Other chimeras include “wingmers,” also known in the art as “hemimers,” that is, oligonucleotides with two distinct regions. In a preferred example of a wingmer, the 5′ portion of the oligonucleotide serves as a substrate for RNase H and is preferably composed of 2′-deoxynucleotides, whereas the 3′ portion is modified in such a fashion so as to have greater affinity for the target RNA molecule but is unable to support nuclease activity (e.g., 2′-fluoro- or 2′-O-methoxyethyl-substituted), or vice-versa. In one embodiment, the oligonucleotides of the present invention contain a 2′-O-methoxyethyl (2′-O—CH₂CH₂OCH)₃ modification on the sugar moiety of at least one nucleotide. This modification has been shown to increase both affinity of the oligonucleotide for its target and nuclease resistance of the oligonucleotide. According to the invention, one, a plurality, or all of the nucleotide subunits of the oligonucleotides of the invention may bear a 2′-O-methoxyethyl (—O—CH₂CH₂OCH₃) modification. Oligonucleotides comprising a plurality of nucleotide subunits having a 2′-O-methoxyethyl modification can have such a modification on any of the nucleotide subunits within the oligonucleotide, and may be chimeric oligonucleotides. Aside from or in addition to 2′-O-methoxyethyl modifications, oligonucleotides containing other modifications which enhance antisense efficacy, potency or target affinity are also preferred. Chimeric oligonucleotides comprising one or more such modifications are presently preferred.

The oligonucleotides used in accordance with this invention may be conveniently and routinely made through the well-known technique of solid phase synthesis. Equipment for such synthesis is sold by several vendors including Applied Biosystems. Any other means for such synthesis may also be employed; the actual synthesis of the oligonucleotides is well within the talents of the routine experimenter. It is well known to use similar techniques to prepare oligonucleotides such as the phosphorothioates and 2′-alkoxy or 2′-alkoxyalkoxy derivatives, including 2′-O-methoxyethyl oligonucleotides (Martin, P., Helv. Chim. Acta 1995, 78, 486-504). It is also well known to use similar techniques and commercially available modified amidites and controlled-pore glass (CPG) products such as biotin, fluorescein, acridine or psoralen-modified amidites and/or CPG (available from Glen Research, Sterling, Va.) to synthesize fluorescently labeled, biotinylated or other conjugated oligonucleotides.

The antisense compounds of the present invention include bioequivalent compounds, including pharmaceutically acceptable salts and prodrugs. This is intended to encompass any pharmaceutically acceptable salts, esters, or salts of such esters, or any other compound which, upon administration to an animal including a human, is capable of providing (directly or indirectly) the biologically active metabolite or residue thereof Accordingly, for example, the disclosure is also drawn to pharmaceutically acceptable salts of the nucleic acids of the invention and prodrugs of such nucleic acids. “Pharmaceutically acceptable salts” are physiologically and pharmaceutically acceptable salts of the nucleic acids of the invention: i.e., salts that retain the desired biological activity of the parent compound and do not impart undesired toxicological effects thereto (see, for example, Berge et al., “Pharmaceutical Salts,” J. of Pharma Sci. 1977, 66, 1-19).

For oligonucleotides, examples of pharmaceutically acceptable salts include but are not limited to (a) salts formed with cations such as sodium, potassium, ammonium, magnesium, calcium, polyamines such as spermine and spermidine, etc.; (b) acid addition salts formed with inorganic acids, for example hydrochloric acid, hydrobromic acid, sulfuric acid, phosphoric acid, nitric acid and the like; salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (d) salts formed from elemental anions such as chlorine, bromine, and iodine.

The oligonucleotides of the invention may additionally or alternatively be prepared to be delivered in a “prodrug” form. The term “prodrug” indicates a therapeutic agent that is prepared in an inactive form that is converted to an active form (i.e., drug) within the body or cells thereof by the action of endogenous enzymes or other chemicals and/or conditions. In particular, prodrug versions of the oligonucleotides of the invention are prepared as SATE [(S-acetyl-2-thioethyl) phosphate] derivatives according to the methods disclosed in WO 93/24510.

For therapeutic or prophylactic treatment, oligonucleotides are administered in accordance with this invention. Oligonucleotide compounds of the invention may be formulated in a pharmaceutical composition, which may include pharmaceutically acceptable carriers, thickeners, diluents, buffers, preservatives, surface active agents, neutral or cationic lipids, lipid complexes, liposomes, penetration enhancers, carrier compounds and other pharmaceutically acceptable carriers or excipients and the like in addition to the oligonucleotide. Such compositions and formulations are comprehended by the present invention.

Pharmaceutical compositions comprising the oligonucleotides of the present invention may include penetration enhancers in order to enhance the alimentary delivery of the oligonucleotides. Penetration enhancers may be classified as belonging to one of five broad categories, i.e., fatty acids, bile salts, chelating agents, surfactants and non-surfactants (Lee et al., Critical Reviews in Therapeutic Drug Carrier Systems 1991, 8, 91-192; Muranishi, Critical Reviews in Therapeutic Drug Carrier Systems 1990, 7, 1-33). One or more penetration enhancers from one or more of these broad categories may be included. Various fatty acids and their derivatives which act as penetration enhancers include, for example, oleic acid, lauric acid, capric acid, myristic acid, palmitic acid, stearic acid, linoleic acid, linolenic acid, dicaprate, tricaprate, recinleate, monoolein (a.k.a. 1-monooleoyl-rac-glycerol), dilaurin, caprylic acid, arachidonic acid, glyceryl 1-monocaprate, 1-dodecylazacycloheptan-2-one, acylcarnitines, acylcholines, mono- and di-glycerides and physiologically acceptable salts thereof (i.e., oleate, laurate, caprate, myristate, palmitate, stearate, linoleate, etc.) (Lee et al., Critical Reviews in Therapeutic Drug Carrier Systems 1991, page 92; Muranishi, Critical Reviews in Therapeutic Drug Carrier Systems 1990, 7, 1; El-Hariri et al., J. Pharm. Pharmacol. 1992 44, 651-654).

In another preferred embodiment, double-stranded RNAs (dsRNAs) are used for reducing the expression of GABA transporters using RNA interference mechanism (Hannon G J. Nature. 2002 418:244-51). RNA interference (RNAi) represents an evolutionary conserved cellular defense mechanism for controlling the expression of alien genes in filamentous fungi, plants, and animals. It is caused by sequence-specific mRNA degradation, and is mediated by dsRNA homologous in sequence to the target RNA. dsRNA is often a byproduct of viral replication or is formed by aberrant transcription from genetic elements after random integration in the host genome. dsRNA is processed to duplexes of 21-nt small interfering RNAs (siRNAs), which guide sequence-specific degradation of the homologous mRNA.

Gene-specific inhibition of gene expression by double-stranded ribonucleic acid (dsRNA) is disclosed for example in U.S. Pat. No. 6,506,559. This patent is incorporated herein in its entirety by reference. The following text includes non-limitative examples for such methods and terminology.

The double-stranded structure may be formed by a single self-complementary RNA strand or two complementary RNA strands. RNA duplex formation may be initiated either inside or outside the cell. The RNA may be introduced in an amount which allows delivery of at least one copy per cell. Higher doses (e.g., at least 5, 10, 100, 500 or 1000 copies per cell) of double-stranded material may yield more effective inhibition; lower doses may also be useful for specific applications. Inhibition is sequence-specific in that nucleotide sequences corresponding to the duplex region of the RNA are targeted for genetic inhibition.

RNA containing a nucleotide sequences identical to a portion of the target gene are preferred for inhibition. RNA sequences with insertions, deletions, and single point mutations relative to the target sequence have also been found to be effective for inhibition. Thus, sequence identity may optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Greater than 90% sequence identity, or even 100% sequence identity, between the inhibitory RNA and the portion of the target gene is preferred. Alternatively, the duplex region of the RNA may be defined functionally as a nucleotide sequence that is capable of hybridizing with a portion of the target gene transcript (e.g., 400 mM NaCl, 40 mM PIPES pH 6.4, 1 mM EDTA, 50° C. or 70° C. hybridization for 12-16 hours; followed by washing). The length of the identical nucleotide sequences may be at least 25, 50, 100, 200, 300 or 400 bases.

As disclosed herein, 100% sequence identity between the RNA and the target gene is not required to practice the present invention. Thus the invention has the advantage of being able to tolerate sequence variations that might be expected due to genetic mutation, strain polymorphism, or evolutionary divergence.

RNA may be synthesized either in vivo or in vitro. Endogenous RNA polymerase of the cell may mediate transcription in vivo, or cloned RNA polymerase can be used for transcription in vivo or in vitro. For transcription from a transgene in vivo or an expression construct, a regulatory region (e.g., promoter, enhancer, silencer, splice donor and acceptor, polyadenylation) may be used to transcribe the RNA strand (or strands). Inhibition may be targeted by specific transcription in an organ, tissue, or cell type; stimulation of an environmental condition (e.g., infection, stress, temperature, chemical inducers); and/or engineering transcription at a developmental stage or age. The RNA strands may or may not be polyadenylated; the RNA strands may or may not be capable of being translated into a polypeptide by a cell's translational apparatus. RNA may be chemically or enzymatically synthesized by manual or automated reactions. The RNA may be synthesized by a cellular RNA polymerase or a bacteriophage RNA polymerase (e.g., T3, T7, SP6). The use and production of an expression construct are known in the art (see WO 97/32016; U.S. Pat. Nos. 5,593,874, 5,698,425, 5,712,135, 5,789,214, and 5,804,693; and the references cited therein). If synthesized chemically or by in vitro enzymatic synthesis, the RNA may be purified prior to introduction into the cell. For example, RNA can be purified from a mixture by extraction with a solvent or resin, precipitation, electrophoresis, chromatography, or a combination thereof. Alternatively, the RNA may be used with no or a minimum of purification to avoid losses due to sample processing. The RNA may be dried for storage or dissolved in an aqueous solution. The solution may contain buffers or salts to promote annealing, and/or stabilization of the duplex strands.

RNA may be directly introduced into the cell (i.e., intracellularly); or introduced extracellularly into a cavity, interstitial space, into the circulation of an organism, introduced orally, or may be introduced by bathing an organism in a solution containing the RNA. Methods for oral introduction include direct mixing of the RNA with food of the organism, as well as engineered approaches in which a species that is used as food is engineered to express the RNA, then fed to the organism to be affected. Physical methods of introducing nucleic acids, for example, injection directly into the cell or extracellular injection into the organism, may also be used. Vascular or extravascular circulation, the blood or lymph system, the phloem, the roots, and the cerebrospinal fluid are sites where the RNA may be introduced.

Physical methods of introducing nucleic acids include injection of a solution containing the RNA, bombardment by particles covered by the RNA, soaking the cell or organism in a solution of the RNA, or electroporation of cell membranes in the presence of the RNA. A viral construct packaged into a viral particle would accomplish both efficient introduction of an expression construct into the cell and transcription of RNA encoded by the expression construct. Other methods known in the art for introducing nucleic acids to cells may be used, such as lipid-mediated carrier transport, chemical-mediated transport, such as calcium phosphate, and the like. Thus the RNA may be introduced along with components that perform one or more of the following activities: enhance RNA uptake by the cell, promote annealing of the duplex strands, stabilize the annealed strands, or otherwise increase inhibition of the target gene.

This invention also provides a pharmaceutical composition which comprises an effective amount of an antibody, preferably a monoclonal antibody, directed to an epitope of the mammalian, preferably human GABA transporter, which is effective in blocking the binding of naturally occurring substrates to the transporter. A monoclonal antibody directed to an epitope of a mammalian GAT1 present on the surface of a cell is disclosed for example in U.S. Pat. No. 6,225,115 and is useful for the purpose of the present invention.

The pharmaceutical composition of the present invention further comprising a pharmaceutically acceptable carrier or diluents. It will be recognized by one of skill in the art that the form and character of the pharmaceutically acceptable carrier or diluent is dictated by the amount of active ingredient with which it is to be combined, the route of administration and other well-known variables. An inhibitor of GABA uptake or a pharmaceutically acceptable salt or hydrate or solvate thereof is administered to a mammal, including a human, in an amount sufficient to prevent or alleviate the symptoms associated with MDMA or MDMA-related drugs.

The route of administration of the pharmaceutical composition is not critical but is usually oral or by injection, preferably oral. Forms of parenteral administration include transdermal, intravaginal or intraperitoneal administration. The subcutaneous and intramuscular forms of parenteral administration are generally preferred. The daily parenteral dosage regimen will be an efficacious, nontoxic quantity preferably selected from the range of about 0.001 mg/kg to about 20 mg/kg of total body weight, most preferably, from about 0.01 mg/kg to about 5 mg/kg. Preferably, each parenteral dosage unit will contain the active ingredient in an amount of from about 2 mg to about 150 mg.

The GABA uptake inhibitors which are active when given orally can be formulated as liquids, for example, syrups, suspensions or emulsions, tablets, capsules and lozenges.

A liquid formulation will generally consist of a suspension or solution of the compound or pharmaceutically acceptable salt in a suitable liquid carrier(s) for example, ethanol, glycerine, non-aqueous solvent, for example polyethylene glycol, oils, or water with a suspending agent, preservative, flavoring of coloring agent.

A composition in the form of a tablet can be prepared using any suitable pharmaceutical carrier(s) routinely used for preparing solid formulations. Examples of such carriers include magnesium stearate, starch, lactose, sucrose and cellulose.

A composition in the form of a capsule can be prepared using routine encapsulation procedures. For example, pellets containing the active ingredient can be prepared using standard carriers and then filled into a hard gelatin capsule; alternatively, a dispersion or suspension can be prepared using any suitable pharmaceutical carrier(s), for example aqueous gums, celluloses, silicates or oils and the dispersion or suspension then filled into a soft gelatin capsule.

The daily oral dosage regimen will be an efficacious, nontoxic quantity preferably selected from the range of about 0.001 mg/kg to about 20 mg/kg of total body weight. Preferably each oral dosage unit will contain the active ingredient in an amount of from about 2 mg to about 150 mg. While it is possible for an active ingredient to be administered alone, it is preferable to present it as a pharmaceutical formulation.

It will be recognized by one of skill in the art that the optimal quantity and spacing of individual dosages of an inhibitor of GABA uptake or a pharmaceutically acceptable salt or hydrate or solvate thereof will be determined by the nature and extent of the exact condition being treated, the form, route and site of administration, and the particular patient being treated, and that such optimums can be determined by conventional techniques. It will also be appreciated by one of skill in the art that the optimal course of treatment, i.e., the number of doses of a GABA uptake inhibitor or a pharmaceutically acceptable salt or hydrate or solvate thereof given per day and duration of therapy, can be ascertained by those skilled in the art of using conventional course of treatment determination tests.

The pharmaceutically acceptable carrier may be of any acceptable form. Examples include, but are not limited to, aqueous physiologically balanced solutions, artificial lipid-containing substrates, natural lipid-containing substrates, oils, esters, glycol, viruses and metal particles.

According to one embodiment of the present invention, the pharmaceutically acceptable carrier includes a delivery vehicle that delivers the nucleic acid sequences to the mammal. Suitable delivery vehicles include, but are not limited to, liposomes, micelles, and cells.

The construction, operation and use of the above pharmaceutically acceptable carriers and the above delivery vehicles are described in detail in U.S. Pat. No. 5,705,151 to Dow et al., entitled “gene therapy for T cell regulation”, which is directed at anti-cancer treatment, and is hereby incorporated by reference as if fully set forth herein.

For therapeutic or prophylactic treatment, the composition according to the present invention may include thickeners, carriers, buffers, diluents, surface active agents, preservatives, and the like, all as well known in the art. Pharmaceutical compositions may also include one or more active ingredients, such as, but not limited to, anti-inflammatory agents, anti-microbial agents, anesthetics and the like.

The following examples are presented in order to more fully illustrate certain embodiments of the invention. They should in no way, however, be construed as limiting the broad scope of the invention. One skilled in the art can readily devise many variations and modifications of the principles disclosed herein without departing from the scope of the invention.

### EXAMPLES

Materials and Methods

Tri Reagent was purchased from MRC Inc., Cincinnati, Ohio, pGEM-T Easy Vector, RNasin and RNase-free DNase I from Promega, Madison, Ill., Superscript™ amplification kit from GIBCOL BRL, Gaitersburg, Md., ethidium bromide and Taq DNA polymerase from Sigma, St. Louis, Mo., and DNA extraction kit from Biological Industries, Israel. LightCycler-FastStart DNA Master SYBR Green I was from Roche Molecular Biochemicals, Mannheim, Germany, Plasmid mini kit from Qiagen, Valencia, Calif., rabbit polyclonal anti-GABA transporter-1 antibodies from Chemicon, Temecula, Calif., and ³³P-[dATP] (3000Ci/mM), nitrocellulose membranes, horseradish peroxidase coupled to F(ab)₂ anti-rabbit IgG, and ECL Western blotting detection reagents from Amersham, Buckinghamshire, England. MDMA was extracted as described in Simantov and Tauber, 1997 from pellets kindly supplied by Dr. R. Levy, Israel Police, Department of Forensic Identification. All other chemicals were of analytical grade.

Animal and Drug Treatment

Young adult male C57BI/6J mice (2-3 months old) were injected intraperitoneally with 0.25 ml saline (control) or saline containing MDMA (10 mg/kg). Unless otherwise indicated, mice were sacrificed 2 hr after drug treatment and frontal cortex and/or midbrain were rapidly dissected in ice-cold saline. The dissected brain tissue was frozen on dry ice, 0.5 ml Tri Reagent was added, and samples were stored at −70° C. Serotonin transporter knockout mice (−/−) (Bengel et al., 1998) were used when indicated.

RNA Preparation, DD-PCR Analysis and cDNA Cloning

Frontal cortex or midbrain samples frozen with Tri Reagent were crushed, 1 ml Tri Reagent was added and total RNA was isolated according to the manufacture's protocol. RNA samples were treated with RNase-free DNase I for 30 minutes at 37° C. and RNA quality and yield was verified on 1.2% formaldehyde agarose gel. First-strand cDNA was prepared from 0.5 μg total RNA as template and 0.8 μM (dT)₁₂GC, (dT)₁₂CC or oligo(dT)₁₈ as a primer, using Superscript amplification kit. DD-PCR (differential display-PCR) was carried out in 20 μl total volume, and included 0.8 μM of one of the 3′ primers (dT)₁₂GC, (dT)₁₂CC or (dT)₁₂, the arbitrary 5′ primer 5′-GGACAGCTTC-3′, 2.5 μM dNTP, 2.5 mM MgCl₂, 2 μCi α-³³P-[dATP] (3000 Ci/mM), cDNA prepared from 50 ng total RNA, and 2.5 units of Taq DNA polymerase. The DD-PCR reaction was started with 3 minutes incubation at 94° C., followed with 40 cycles of 30 sec at 94° C., 2 minutes at 40° C. and 30 sec at 72° C., and finally 5 minutes at 72° C. Then, 10 μl of the reaction was mixed with 10 μl of 95% formamide containing 0.05% bromophenol blue and 0.05% xylene cyanol, and incubated for 5 minutes at 75° C. An aliquot (6 μl) was analysed on a denatured DNA sequencing gel. The gel was dried and exposed to Phosphoimage Analyzer (Fujix) or to X-ray film. The DNA bands expressed differentially upon MDMA-treatment were identified, cut from the gel, socked in H₂O, boiled for 15 minutes and centrifuged (10000 g). Ten μl of the supernatant was used as template to re-amplify the band of interest, with the same primers and PCR conditions described for DD-PCR except that 25 μM dNTP and no isotopes were used. Ten μl of the PCR reaction was run on a 2% agarose gel. The band of the predicted size was excised, purified with DNA extraction kit, and cloned into pGEM-T Easy Vector. Plasmid DNA from successful clones was purified with Plasmid Mini Kit and subjected for sequencing (Biological Services, Weizmann Institute of Science). Sequence data were compared with GeneBank (NCBI).

RT-PCR: Primers, Reaction Conditions and Semi-Quantitation

Total RNA was isolated, treated with DNase, and first-strand cDNA was synthesized from 0.2-1.0 μg total RNA, using the arbitrary primer 5′-GGACAGCTTC-3′ and the Superscript amplification kit. PCR was carried out in 20 μl including 0.5 μM of 3′- and 5′ primers indicated for each gene, 25 μM dNTP, 2.5 mM MgCl₂, cDNA prepared from 10-100 ng total RNA, 5 units RNasin, and 2.5 units Taq DNA polymerase. The PCR reaction was started with 3 minutes of incubation at 94° C., followed by 25-33 cycles of 30 sec at 94° C., 45 sec of annealing at temperatures indicated for each gene (Table 1), 30 sec at 72° C., and finally 7 minutes at 72° C. Primers were designed according to the sequence of the cloned cDNAs or the reported sequences of mGAT1, mGAT2, mGAT4 and glyceraldehyde-3-phosphate dehydrogenase (GAPDH), using the Genetics Computer Group (GCG) Prime program. mGAT1, mGAT2 and mGAT4 primer sequences (SEQ ID NOS. 12, 14 AND 15, respectively) correspond to nucleotides 2535-2554 and 2821-2840, 187-206 and 349-348, and 2347-2356 and 2529-2548, respectively (Lopez-Corcuera et al., 1992; Liu et al., 1993). Table 1 shows the primers' sequences, annealing temperatures and size of DNA products. Semi-quantitation of RT-PCR results was performed by comparison with GAPDH, using NIH Image Analysis Program or the Histogram setting of the Adobe Photoshop 4.0.

TABLE 1

RT-PGR primers for 3 GATs and 3 genes
identified, annealing temperatures,
and the size of cDNAs products

| | | AT | cDNA |
|---|---|---|---|
| Gene/DNA | Primer Sequence | (° C) | Product (bp) |
| mGAT1 | F: 5′TAACAACAACAGCCCAT | 60 | 326 |
| | CCA 3′ | | |
| | (SEQ ID NO 12) | | |
| | R: 5′GGAGTAACCCTGCTCGA | | |
| | TGA3′ | | |
| | (SEQ ID NO 13) | | |
| mGAT2 | F: 5′GAGTTTGTGCTGTCAGT | 60 | 182 |
| | GGC3′ | | |
| | (SEQ ID NO 14) | | |
| | R: 5′CTCCCCTGGCTGCTATA | | |
| | CTG3′ | | |
| | (SEQ ID NO 15) | | |
| mGAT4 | F: 5′TTTGGTCTTCCCCTTTT | 60 | 214 |
| | CCT3′ | | |
| | (SEQ ID NO 16) | | |
| | R: 5′AAGACTCCACTCAACCC | | |
| | CCT 3′ | | |
| | (SEQ ID NO 17) | | |
| MC2 | F: 5′GGAGGGGTCTAAGAGCA | 68 | 228 |
| | TCCGAAA3′ | | |
| (SEQ ID NO 2) | (SEQ ID NO 18) | | |
| | R: 5′AAGATTGGAAGAGCCAC | | |
| | AGCCCAT3′ | | |
| | (SEQ ID NO 19) | | |
| MG2 | F: 5′AAGACGTTTGAGGGATC | 56 | 212 |
| (SEQ ID NO 3) | TTT3′ | | |
| | (SEQ ID NO 20) | | |
| | R: 5′TTAAGGGGTGATCACAA | | |
| | TTC3′ | | |
| | (SEQ ID NO 21) | | |
| MG4 | F: 5′TGAGATGACGCACGGTT | 60 | 247 |
| (SEQ ID NO 5) | AAG3′ | | |
| | (SEQ ID NO 22) | | |
| | R: 5′GAAAACTTACCTCCTCG | | |
| | CCC3′ | | |
| | (SEQ ID NO 23) | | |
| GAPDH | F: 5′GAAGATGATCCCTGGCT | 58 | 294 |
| | CTAGTGG3′ | | |
| | (SEQ ID NO 24) | | |
| | R: 5′AATGCCAGCCCGAGCGT | | |
| | CAAAGGT3′ | | |
| | (SEQ ID NO 25) | | |

AT—annealing temperature. F—forward primer, R—reverse primer. bp—base pair. Primers for mGAT1, mGAT2 and mGAT4 were designed as described in Materials and Methods. GAPDH was used as a standard.

Real-Time Quantitative PCR Analysis

Real-time PCR analysis was performed with the LightCycler System (Roche Molecular Biochemicals) and LightCycler-FastStart DNA Master SYBR Green I, according to the manufacturer instructions. Reactions (in 20 μl) included template cDNA prepared from 0.1-0.5 μg total RNA, 3 mM MgCl₂ and 0.25-0.5 μM primers. Buffer, dNTP mixture (with dUTP instead of dTTP), Taq DNA polymerase, and SYBR Green I dye were included in the ‘Hot start’ reaction as recommended. Typical protocol was as follows. Ten minutes at 95° C. followed by 40 cycles of amplification, starting with 15 sec at 95° C., 10 sec at 60° C. and 10 sec at 72° C. The extension period varied with each specific primer, depending on the product length (about 1 sec/25 bp).

For quantification analysis, 3-5 standards of PCR products with known concentration were amplified under the same cycling conditions, and GAPDH was amplified as an internal control to correct for variations in the amount of cDNA. Real-time PCR products of each assay were also subjected to agarose gel electrophoresis, to further confirm amplification specificity. Quantitation was assessed with LightCycler Data Analysis (LCDA) software by the Fit Points method. Melting curve of the cDNA product was determined by incubation at 95° C. for 0 sec, 15 sec at 65° C. and 0 sec at 95° C., followed by a cooling of 30 sec at 40° C.

Western Immunoblotting with Anti-GAT1 Antibodies

Protein was isolated with Tri Reagent according to the protocol provided by the manufacturer. After precipitation with isopropanol, the protein pellet was washed three times with 0.3M guanidine hydrochloride and 95% ethanol, followed by washing with ethanol, dried, and dissolved in 10 M urea containing 50 mM dithiothreitol (DTT). Samples were kept at room temperature for 1 hr before boiling for 3 minutes, sonicated on ice for 2 minutes, and stored at −20° C. Protein concentration was determined with the micro BCA protein assay reagent kit. Samples of 30 μg protein run on SDS-PAGE (5% stacking gel and 8% or 10% separating gel), with Kaleidoscope pre-stained standard proteins as molecular weight markers. The proteins were transferred to nitrocellulose membranes preincubated for 2 hr in Tris-buffered saline (TBS) with 10% non-fat dry milk and 0.1% Tween 20, and incubated overnight at 4° C. or 2 hr at room temperature with anti-GAT1 antibodies (1:500 in TBS containing 5% non-fat dry milk). Membranes were then washed three times with TBS containing 0.1% Tween 20, incubated for 2 hr at room temperature with the second antibody, horseradish peroxidase coupled to F(ab)₂ anti-rabbit IgG, washed four times with the same buffer, and subjected to ECL Western immublotting detection reagents.

### Example 1

### Altered mRNA Expression upon MDMA Treatment: Cloning of 11 cDNAs Expressed Differentially

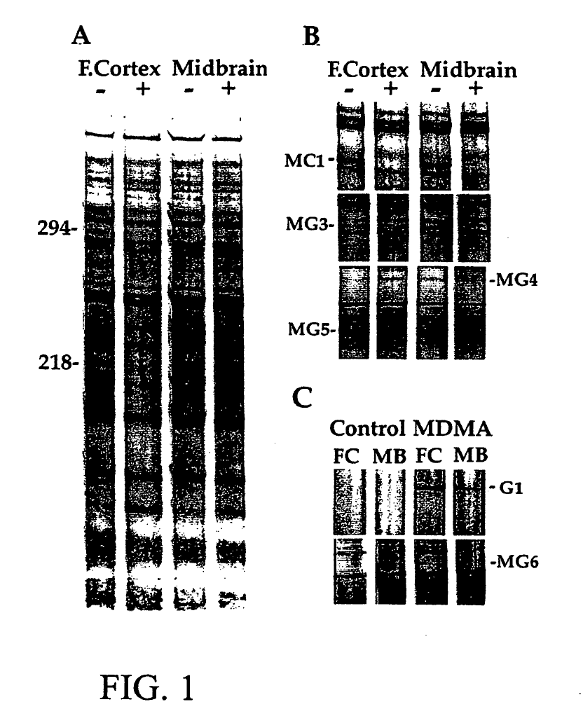

Total RNA from frontal cortex (“FC”) and midbrain (“MB”) of saline—(denoted as “−”) and MDMA-treated (denoted as “+”) mice (one i.p. injection, 10 mg/kg, 2 hr) were used for DD-PCR analysis. The random primers used were dT₁₂CC for MC1, dT₁₂GG for MG3, MG4, MG5 and MG6, and dT₁₈ for G1. More information about these and the other five cDNAs isolated, cloned and sequenced is shown in Table 2. Different but characteristic cDNA products were obtained with each one of the three primers. FIG. 1A depicts the complete cDNA profile of a representative sequencing gel, demonstrating that MDMA treatment altered the intensity of a number of cDNAs, sometimes increased and others decreased from control. Some of the altered expression levels are common for both brain regions while others are specific either to the frontal cortex or the midbrain.

FIGS. 1B and 1C, demonstrate increased or decreased expression of few cDNAs. For example, both the frontal cortex (“FC”) and midbrain (“MB”) of MDMA-treated mice showed a profound decrease in the cDNA band named MC1 (FIG. 1B), whereas the intensity of the cDNA G1 was increased (FIG. 1C).

Overall, we report herein the isolation, cloning and sequencing of 11 cDNAs, expression of which was altered upon MDMA treatment (Table 2). The size of these cloned DNA fragments ranged between 218-498 bp, and the GeneBank analysis showed that four of them are at least 98% identical to previously cloned genes. The sequence of another five of the cloned cDNAs are similar to previously isolated ESTs, and the remaining cDNAs, named MC1 and MC2 are novel. Table 2 summarizes the effect of MDMA treatment on the expression of these 11 cDNAs, with expression of 5 of them increasing and 6 decreasing. Intensity of each band was designated according to a scale from − (lowest) to ++ (highest). No score indicates that the analysis was performed from only the other brain region. GeneBank accession numbers for C3, C4, C5 and G1, were U10355, M68859, D49382 and M92378, respectively.

TABLE 2
Genes and cDNAs identified by DD-PCR
analysis: Effect of MDMA

| CDNA | Control | | MDMA | | |
|---|---|---|---|---|---|
| Fragment | F. | | F. | | Blastn |
| (bp) | Cortex | MidBrain | Cortex | MidBrain | % Homology |
| C3 (356) | ++ | | + | | 98 |
| C4 (498) | | ++ | | + | 99.4 |
| C5 (421) | | ++ | | + | 99.5 |
| G1 (298) | − | − | ++ | + | 99.3 |
| MG2 (349) | − | | ++ | | EST |
| MG3 (298) | + | + | − | − | EST |
| MG4 (399) | − | − | + | + | EST |
| MG5 (388) | + | + | − | − | EST |
| MG6 (399) | − | − | + | + | EST |
| MC1 (218) | ++ | ++ | − | − | Unknown |
| MC2 (448) | | + | | ++ | Unknown |

### Example 2

### MDMA Regulates Expression of GABA Transporters in a Selective Way

A comparison with Genebank data showed that one of the isolated cDNAs, G1, has 99.3% identical sequence to the mouse GABA transporter 1 (mGAT1) (Lopez-Corcuera et al., 1992). In light of previous reports indicating some relationship between serotonergic and GABAergic systems in the brain, we decided to focus on this gene family. First, it was desirable to verify the effect of MDMA administration on the expression of mGAT1 by RT-PCR analysis, using specific primers designed according to the mouse mGAT1 sequence reported by others (Lopez-Corcuera et al., 1992). Sequence of the primers, annealing temperatures and the size of DNA products is shown in Table 1. GAPDH levels were determined for comparison. Data are representative from experiments replicated 2-4 times.

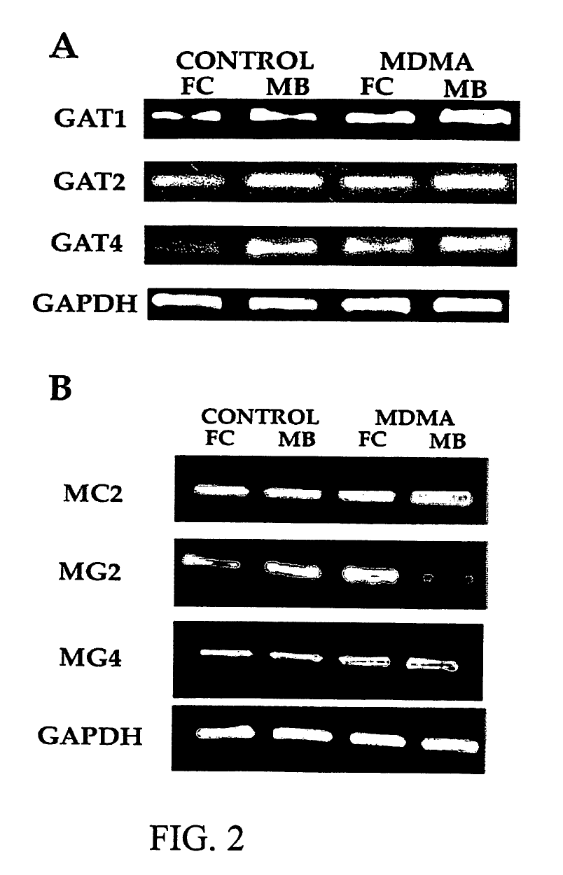

As demonstrated in FIG. 2A, MDMA increased the expression of mGAT1 in the frontal cortex and midbrain, estimated by scanning to be 1.58 and 1.74 fold, respectively. Similar RT-PCR analysis of three other isolated cDNAs, MC2, MG2 and MG4 (FIG. 2B) also confirmed the MDMA-induced changes observed by the DD-PCR approach, described in Table 2 in a range of from about 1.5-2.2 fold.

The effect of MDMA administration on mRNA expression of two other GABA transporters that are abundant in adult brain, mGAT2 and mGAT4 was determined by RT-PCR, using specific primers. It appears that MDMA had insignificant effect on mGAT2 expression, whereas the level of mGAT4 mRNA was increased in the frontal cortex and midbrain by 4.6 and 1.7 fold, respectively (FIG. 2A). GAPDH was used for semi-quantitation. The mGAT3 subtype was not studied in the current work as it is mostly expressed in neonatal brain (Liu et al., 1993).

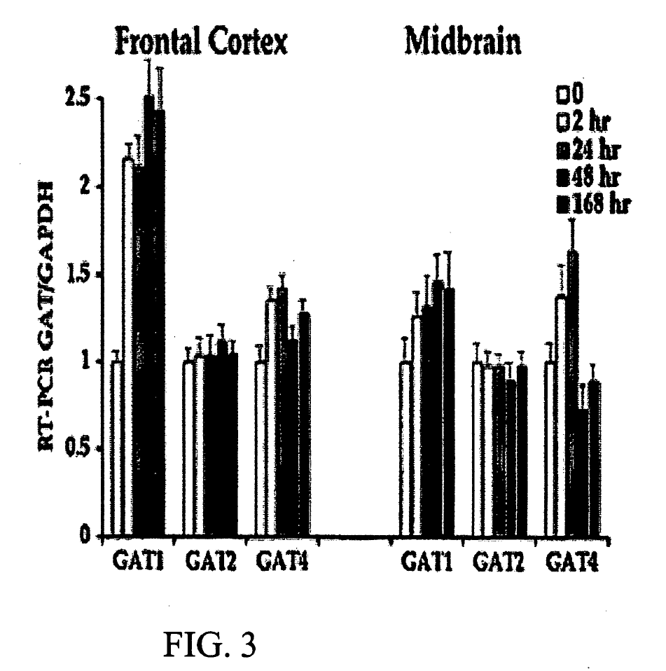

As demonstrated in FIG. 3, a time course experiment was performed to determine whether the effects of MDMA administration on expression of mGAT1 and mGAT4 was transient or lasted for several days, and whether mGAT2 expression was altered at time points later than 2 hours. Data are mean ±SD from an experiment replicated twice. RT-PCR analysis of the three GABA transporters shows that MDMA effect on mGAT1 sustained for 7 days, whereas mGAT2 expression did not change from 2 hours to 7 days after treatment. Unlike the effect on mGAT1, the increase in mGAT4 was transient, as it returned partially (frontal cortex) or completely (midbrain) to control levels within 2 days.

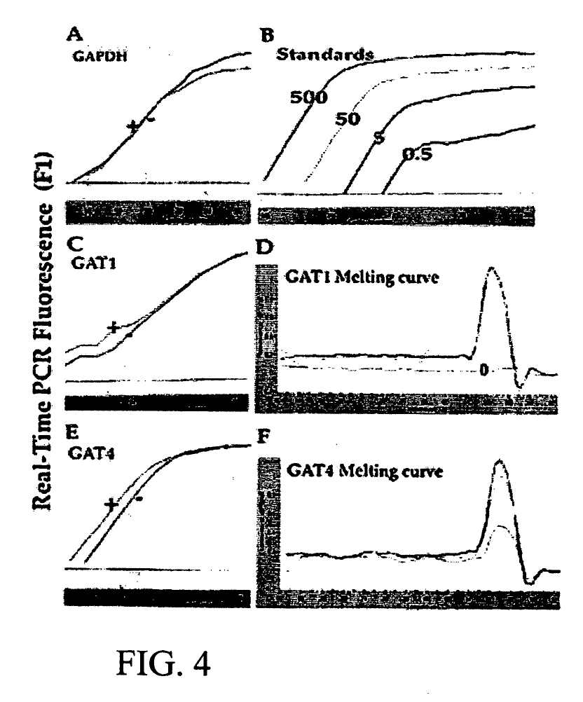

As demonstrated in FIG. 4, real-time PCR analysis was used to further verify MDMA effect on expression of mGAT1 and mGAT4, and to obtain quantitative data. RNA extracted from the midbrain of saline- or MDMA-treated mice was analyzed with the LightCycler System, using specific primers for mGAT1 and mGAT4, along with GAPDH as a control (see Materials and Methods). As shown in FIG. 4C, amplification of mGAT1 revealed that treatment with MDMA increased the expression of this gene from 3.43×10⁻⁵ to 21.1×10⁻⁵ pmole. Under the same conditions GAPDH level was increased by 19% (from 514×10⁻⁵ to 612×10⁻⁵ pmole), indicating a net increase of 5.2 fold in mGAT1 levels. As shown in FIG. 4D, the melting curve profile of mGAT1 PCR products of both control and MDMA-treated samples displayed a single peak at 86.3° C. As shown in FIG. 4D, similar real-time PCR analysis of mGAT4 showed a 1.52 fold increase in mGAT4/GAPDH ratio in MDMA treated midbrain as compared with controls (FIG. 4E), with a single melting curve peak at 86.6±0.2° C. (FIG. 4F).

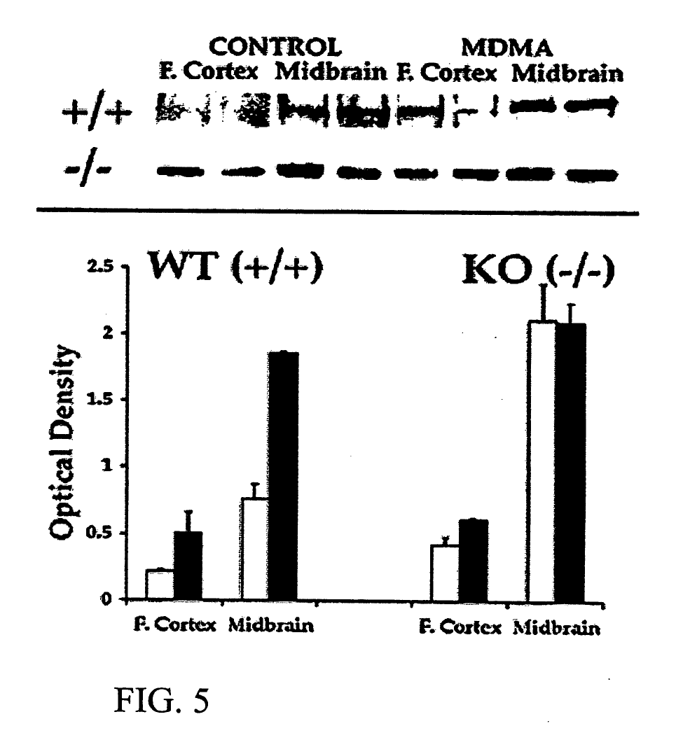

It was essential to determine whether the MDMA-induced increase in mGAT1 mRNA was correlated with a similar change in protein level. Western immunoblotting (FIG. 5 upper panel) and quantitation by scanning the GAT1 protein band (FIG. 5 lower panel) showed that MDMA treatment increased GAT1 immunoreactivity in the frontal cortex and midbrain by 2.1 and 2.4 fold, respectively. These anti-GAT1 antibodies were also useful to verify MDMA effect on the expression of mGAT1 in the SERT knockout mice (−/−) that behaviorally are nonresponsive to MDMA (Bengel et al., 1998). FIG. 5 shows no significant induction of GAT1 protein in SERT −/− mice upon MDMA treatment (1.3 and 1.0 fold of control frontal cortex and midbrain, respectively).

### Example 3

### Protective Activity of GABA Transporter Inhibitors Against MDMA Acute Toxicity in Mice

Next step was to determine whether compounds blocking the enhanced expression of GABA transporters may restraint MDMA-induced hyperthermia. We ran trials to determine whether the mortality levels causes by MDMA administration would be affected by treatment with suspected GABA transporter inhibiting compounds Tiagabine, nipecotic acid derivative NO-711, and Vigabatrin which have been used hitherto as anti-epileptics.

Tiagabine hydrochloride monohydrate, also known as Gabitril, is reportedly a known potent selective uptake inhibitor of GAT1 in the cortex and hippocampus and has been shown in animal models to be a potential treatment for certain kinds of seizures. It has also been suggested that it may be useful for disorders such as pain.

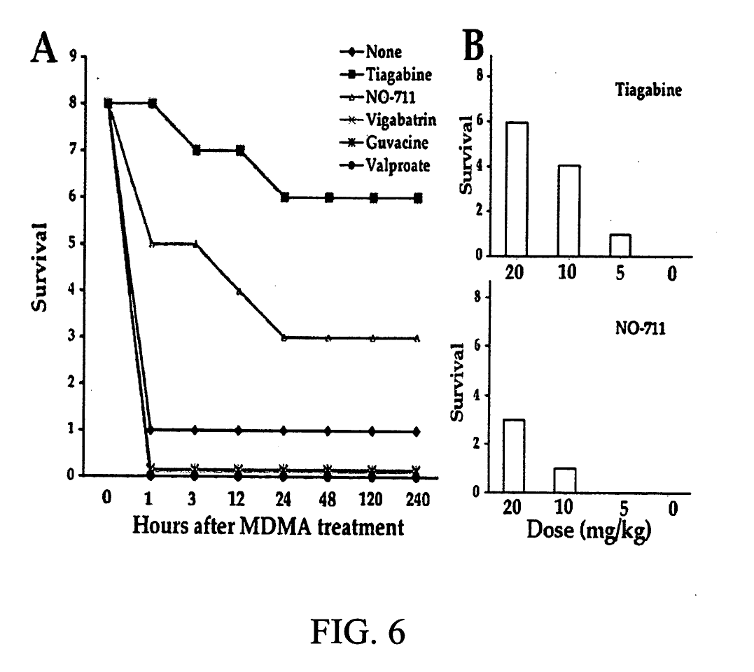

Mice were administered dose(s) of mg/ml of MDMA according to the following schedule and were simultaneously injected with a dose of the GABA transporter blocker. The results on the mortality of the subjects are shown in FIG. 6A-C. In the first set of experiments the effective toxic dose of MDMA was determined. C57B1/6J mice, 2-3 months old, 6 mice in each group, were tested with different concentration of the drug, prepared in saline and injected intraperitoneally. Saline solution was used as a control. The lowest concentration of the drug causing 100% death within 60 minutes was used in the following experiments. Mice (8 in each group) were injected (i.p.) with different concentrations of the tested GABA transporter inhibitor, or other compound, and 5-120 minutes later were injected with the toxic dose of MDMA. GABA transporter inhibitors which we investigated include: NO 711 [1-(2(((diphenylmethylene)imino)oxy)ethyl)-1,2,5,6-tetrahydro-3-pyridinecarboxylic acid hydrochloride]; Tiagabine [(R)-1-[4,4-Bis(3-methyl-2-thienyl)-3-butenyl]-3-piperidinecarboxylic acid}; and Vigabatrin [4-Amino-5-hexenoic acid]. As shown in FIG. 6A, GABA transporter inhibitors Tiagabine and NO-711 significantly improved the survival of MDMA-treated mice. The effective doses of Tiagabine and NO-711 which improve the survival of MDMA-treated mice is demonstrated in FIG. 6B.

### REFERENCES

Bengel D, Murphy D L, Andrews A M, Wichems C H, Feltner D, Heils A, Mossner R, Westphal H, Lesch K P (1998) Altered brain serotonin homeostasis and locomotor insensitivity to 3,4-methylenedioxymethamphetamine (“Ecstasy”) in serotonin transporter-deficient mice. Mol Pharmacol 53:649-655.
Finnegan K T, Taraska T (1996) Effects of glutamate antagonists on methamphetamine and 3,4-methylenedioxymethamphetamine-induced striatal dopamine release in vivo. J Neurochem 66:1949-1958.
Green A R, Cross A J, Goodwin G M (1995) Review of the pharmacology and clinical pharmacology of 3,4-methylenedioxymethamphetamine (MDMA or “Ecstasy”). Psychopharmacology 119:247-260.
Hegadoren K M, Baker G B, Bourin M (1999) 3,4-Methylenedioxy analogues of amphetamine: defining the risks to humans. Neurosci Biobehav Rev 23:539-553.
Liu Q R, Lopez-Corcuera B, Mandiyan S, Nelson H, Nelson N (1993) Molecular characterization of four pharmacologically distinct gamma-aminobutyric acid transporters in mouse brain. J Biol Chem 268:2106-2112.
Lopez-Corcuera B, Liu Q R, Mandiyan S, Nelson H, Nelson N (1992) Expression of a mouse brain cDNA encoding novel gamma-amino-butyric acid transporter. J Biol Chem 267:17491-17493.
Shankaran M, Gudelsky G A (1998) Effect of 3,4-methylenedioxymethamphetamine (MDMA) on hippocampal dopamine and serotonin. Pharmacol Biochem Behav 61:361-366.
Simantov R, Tauber M (1997) The abused drug MDMA (Ecstasy) induced programmed death of human serotonergic cells. FASEB J 11:141-146.
Stephenson C P, Hunt G E, Topple A N, McGregor I S (1999) The distribution of 3,4-methylenedioxymethamphetamine “Ecstasy”-induced c-Fos expression in rat brain. Neuroscience 92:1011-1023.
Yamamoto B K, Nash J F, Gudelsky G A (1995) Modulation of methylenedioxymethamphetamine-induced striatal dopamine release by the interaction between serotonin and gamma-aminobutyric acid in the substantia nigra. J Pharmacol Exp Ther 273:1063-1070.
Zakzanis K K, Young D A (2001) Memory impairment in abstinent MDMA (“Ecstasy”) users: a longitudinal investigation. Neurology 56:966-969.

It will be appreciated by a person skilled in the art that the present invention is not limited by what has been particularly shown and described hereinabove. Rather, the scope of the invention is defined by the claims that follow.

1. A method for treating or preventing the symptoms associated with the ingestion of methylenedioxymethamphetamine (MDMA) or MDMA-related psychoactive drugs which comprises administering to a subject in need thereof a therapeutic composition comprising a therapeutically effective amount of an agent which inhibits γ-amino butyric acid (GABA) reuptake, thereby treating or preventing the symptoms associated with MDMA or MDMA-related psychoactive drugs.

2. The method of claim 1, wherein said agent inhibits GABA reuptake by inhibiting GABA transporter activity or by reducing GABA transporter level in the brain.

3. The method of claim 2, wherein the agent which inhibits GABA transporter activity is a GABA transporter inhibitor or blocker.

4. The method of claim 3, wherein the GABA transporter inhibitor or blocker is selected from the group consisting of Tiagabine, diaryloxime ether substituted tiagabine, diarylvinyl ether substituted tiagabine, Vigabatrin and nipecotic acid derivatives.

5. The method of claim 2, wherein said GABA transporter is GABA transporter 1 (GAT-1) or GABA transporter 4 (GAT-4).

6. The method of claim 2, wherein the agent which reduces GABA transporter level is a nucleic acid molecule capable of reducing the expression of GABA transporter in the brain.

7. The method of claim 6, wherein said GABA transporter is GABA transporter 1 (GAT1) or GABA transporter 4 (GAT4).

8. The method of claim 7, wherein said nucleic acid molecule is an antisense molecule or a double stranded RNA (dsRNA).

9. The method of claim 2, wherein the agent which inhibits GABA transporter activity in the brain is an anti-GABA transporter antibody.

10. The method of claim 1, wherein said symptoms are selected from: psychostimulation, hallucination, hyperthermia, memory loss and long-lasting changes in behavior.

11. The method of claim 1, wherein said subject is a human subject.

12. A pharmaceutical composition for treating or preventing the symptoms associated with MDMA or MDMA-related drugs comprising a therapeutically effective amount of an agent which inhibits GABA reuptake, and a pharmaceutically acceptable carrier.

13. The composition of claim 12, wherein said composition is suitable for administration topically, orally or parenterally.

14. The composition of claim 12, wherein said agent inhibits GABA reuptake by inhibiting GABA transporter activity or by reducing GABA transporter level in the brain.

15. The composition of claim 14, wherein the agent which inhibits GABA transporter activity is selected from the group consisting of Tiagabine, diaryloxime ether substituted tiagabine, diarylvinyl ether substituted tiagabine, Vigabatrin and nipecotic acid derivatives.

16. The composition of claim 15, wherein said GABA transporter is GAT-1 or GAT-4.

17. The composition of claim 16, wherein the agent which reduces GABA transporter level is an antisense oligonucleotide molecule or dsRNA molecule capable of inhibiting the expression of said GABA transporter.

18. The composition of claim 16, wherein the agent which reduces GABA transporter activity is an anti-GABA transporter antibody or a GABA transporter blocker or inhibitor.

19. A composition comprising an expression vector and a pharmaceutically acceptable carrier, wherein said expression vector is capable of expressing an oligonucleotide which is substantially complementary to a nucleic acid molecule encoding GAT-1 or GAT-4.

20. A method of attenuating the hyperthermic effects of MDMA or MDMA-related drugs comprising administering to a subject in need a therapeutic composition comprising a therapeutically effective amount of an agent which inhibits GABA reuptake, thereby attenuating the hyperthermic effects associated with MDMA or MDMA-related drugs.
